(19) United States
(12) Patent Application Publication
(10) Pub. No.: US 2014/0363846 A1
(43) Pub. Date: Dec. 11, 2014

(54) PROCESS FOR PRODUCING CELLULASE MIXTURES FROM MYCELIOPHTHORA AND RELATED ORGANISMS

(71) Applicants: Jason B. Edwards, Alberta (CA); Glenn Munkvold, Ontario (CA)

(72) Inventors: Jason B. Edwards, Alberta (CA); Glenn Munkvold, Ontario (CA)

(73) Assignee: IOGEN ENERGY CORPORATION, Ottawa, ON (CA)

(21) Appl. No.: 14/241,101

(22) PCT Filed: Aug. 24, 2012
(86) PCT No.: PCT/CA12/50585

§ 371 Date: Feb. 26, 2014

Related U.S. Application Data
(60) Provisional application No. 61/527,755, filed on Aug. 26, 2011.

(51) Int. Cl. C12N 9/24; C12P 19/14

(52) U.S. Cl. CPC C12N 9/2402; C12P 19/14; C12Y 302/01004
USPC 435/72; 435/209

(57) ABSTRACT

The present invention provides a fermentation process for producing a cellulase enzyme mixture comprising the steps of (a) providing a fungal cell of the genus Myceliophthora or a taxonomically equivalent genus; (b) culturing the fungal cell in a submerged liquid batch culture in which the carbon source comprises about 0% of cellulase-inducing carbon source; (c) culturing the fungal cell from the batch culture from step (b) in a submerged liquid fed-batch and/or continuous culture; and (d) providing the culture of step (c) with a feed solution containing a about 100 wt % non-inducing carbon source (such as glucose, dextrose, sucrose, molasses, fructose, glycerol, xylose, or a combination thereof), wherein the feed solution is provided at a rate that maintains the concentration of non-inducing carbon source in the culture below that which would otherwise repress production of the cellulase enzyme mixture, so as to produce a culture filtrate containing at least 10 g protein/L.

### FIELD OF THE INVENTION

The present invention relates to a fermentation process for producing cellulases from a fungal host cell.

### BACKGROUND

Cellulose, the most abundant polysaccharide in the biosphere, consists of D-glucose units connected together in linear chains via beta-1,4 glycosidic bonds. The second most abundant biopolymer, after cellulose, is hemicellulose which consists of a linear series of D-xylose units linked together via beta-1,4 glycosidic bonds forming a core structure from which extend numerous sided chains, such as L-arabinose, acetic acid, and ferulic acid, linked to xylose via glycosidic bonds or ester bonds. When considered together, both cellulose and hemicellulose represent a renewable carbon source for the production of fermentable sugars (e.g. glucose, xylose, and arabinose) that is relatively inexpensive.

In nature, microorganisms such as bacteria and fungi produce a suite of enzymes that break down cellulose and hemicellulose into their constituent monosaccharides (e.g. glucose, xylose, and arabinose). Myceliophthora thermophila is a filamentous fungus capable of producing a large variety of cellulases and hemicellulases. M. thermophila also produces accessory enzymes, like beta-glucosidases, which enhance the action of cellulases. Myceliophthora thermophila is known by several other names such as: Chrysosporium thermophilum, Myceliophthora indica, Cornyascus heterothallicus (Maheshwari, R., et al. (2000). Microbiol. Mol. Biol. Rev. 64:461-488). Myceliophthora thermophila is also known as the asexual anamorph of Sporotrichum thermophile and Chrysosporium thermophile (Maheshwari, R. et al., 2000; Canevascini, G., et al., (1983) Can. J. Microbiol. 29: 1071-1080) and Thielavia heterothallica (NCBI Taxonomy ID: 78579). Some strains of Myceliophthora thermophila have been previously identified as Chyrosporium lucknowense (Visser, H. et al., (2011) Industrial Biotech. 7: 214-223).

The regulation of the production of cellulases and hemicellulases is a complex process in Myceliophthora thermophila and filamentous fungi in general. It is believed to be controlled primarily at the transcriptional level in response to signals associated with available carbon sources. There are many instances in which strains synonymous with M. thermophila have been used to produce small quantities of enzymes for laboratory analysis. Coutts and Smith (Coutts, A. D. and Smith, R. E. (1976) Appl. Enviro. Micro. 31: 819-825) showed that flask cultures of S. thermophile (strain UAMH 2015) produced cellulase activity when cellulose (Solka Floc) was used as a carbon source. Canevascini et al. (Canevascini, G., et al., (1979) J. Gen. Micro. 110: 291-303) conducted flask culture experiments to characterize induction and repression of cellulase in wild-type strain of S. thermophile (strain var.2). They showed that flask cultured mycelia that had been washed and transferred to fresh media containing various different kinds of cellulose (e.g. Avicel, fibrous cellulose powder, microgranular cellulose powder, insoluble carboxymethylcellulose, and soluble carboxymethylcellulose) produced substantial cellulase activity within 4 hours; in contrast, cultures of transferred mycelia produced negligible amounts of cellulase activity over the same time period when only glucose was present as a carbon source. Similar experiments using a variety of small molecular weight carbon s identified cellobiose, and to a lesser extent laminaribose, as an inducer of cellulase production. Cultures with gentiobiose, sophorose, maltose, trehalose, mannose, fructose, xylose, sucrose, lactose, or glycerol all produced less than ⅕ the quantity of cellulase activity found in cellobiose cultures; therefore, none of these compounds were considered inducers. In flask culture experiments with multiple carbon sources, glucose concentrations as low as 0.05% w/v were shown to completely inhibit (i.e. repress) the cellulase induction effects of cellobiose.

Roy et al. ((1988) App. Enviro. Micro. 54: 2152-2153) found that the addition of crystalline cellulose (as Solka-Floc) and cellobiose induced beta-glucosidase production in flask cultures of M. thermophila D-14 while addition of glucose to the culture medium severely repressed production of this enzyme. Bhat and Maheshwari (Bhat, K. M. and Maheshwari, R. (1987) Appl. Enviro. Micro. 53: 2175-2182) demonstrated cellulase production in flask cultures by several laboratory mutants of Sporotrichum thermophile (e.g. strains IIS 101, IIS 220, ATCC 42464). They detected endoglucanase, exoglucanase, and beta-glucosidase activities in filtrates of cultures grown with lactose or cellulose as a carbon source. In contrast, S. thermophile filtrates from cultures grown on glucose, maltose, sucrose, starch or sodium carboxymethyl cellulose as a carbon source did not possess these enzyme activities. Canevascini et al. (Canevascini, G., et al. (1983) Can. J. Microbiol. 29: 1071-1080) found that cultures of S. thermophile (ATCC 42464) grown on cellobiose, crystalline cellulose, and amorphous cellulose all produced various proteins including endoglucanases, exoglucanases, and cellobiose dehydrogenase. They noted that the relative proportion of endoglucanases I, II and III changed when cellobiose was used instead of cellulose. It was proposed that cellulases adsorbed to amorphous cellulose and thus altered the concentration and composition of enzymes in the culture filtrate. Bhat and Maheshwari. ((1987) Appl. Enviro. Micro. 53: 2175-2182) identified multiple forms of beta-glucosidase made by S. thermophile (strain IIS 220) in shake flasks with cellulose-containing medium.

U.S. Pat. Nos. 5,811,381, 6,015,707 and 7,892,812 provide examples of producing cellulase from Chrysosporium lucknowense strain C1 (subsequently reclassified as M. thermophila—Visser, H. et al. (2011) Industrial Biotech. 7: 214-223) in shake flasks in which the carbon sources were either a combination of: 1) sweet beet pulp (press), barley malt, and wheat bran, 2) lactose and corn steep liquor, 3) lactose and peptone, 4) cellulose and peptone, 5) cellulose and cornsteep liquor. Cellulase was also produced in a 10 L batch fermentation using lactose as an inducing carbon source. Cellulase production was also reported for 60 L batch and fed-batch fermentations using a combination of yeast extract, lactose, defatted cotton seed flour, and cellulose (Sigmacell 50) as carbon sources. No cellulase production was observed in shake-flask cultures in which a combination of cellulose with any one of glucose, dextrose or glycerol was used as carbon source.

Cellulase production by a related fungal species, Thielavia terrestris, has been reported. U.S. Pat. No. 7,361,495 B2 and U.S. Publication No. 2008/0289067 provide examples of cellulase production by Thielavia terrestris (strain NRRL 8126) in batch cultures, at flask- and pilot fermenter-scale, using cellulose as a carbon source.

Although there are a variety of methods for producing cellulase from M. thermophila and synonymous organisms, they are not necessarily appropriate for industrial use. Industrial scale production of protein seeks to maximize quantity and quality of products while minimizing costs associated with operations (e.g. materials, energy) or capital (e.g. fermentors, pumps, holding tanks). Those skilled in the art will appreciate that working with solid substrates, like cellulose, can be problematic in terms of both maintaining aseptic fermentation conditions as well as handling procedures during upstream and downstream processing. Refined cellulose is also a relatively costly substrate. Low cost, crude cellulose preparations (e.g., from pretreated lignocellulose) can present additional problems associated with quality control and/or the undesirable addition of compounds resulting from the pretreatment process to the fermentation Soluble carbon sources are preferred as both inducers and bulk carbon sources given their ease of handling. Further benefits can be realized if such soluble substrates are more readily sterilizable and less costly than cellulose. Another advantage of using soluble substrates instead of cellulose for cellulase production in particular is that loss of product quantity or quality due to adsorption can be avoided (Canevascini, G., et al. (1983) Can. J. Microbiol. 29: 1071-1080).

### SUMMARY OF THE INVENTION

The present invention relates to a fermentation process for producing cellulase enzyme mixtures from submerged liquid cultures of fungi.

The present invention provides a fermentation process for producing cellulase enzyme mixtures from fungal cells of the genus Myceliophthora and taxonomically equivalent fungi in submerged liquid cultures.

In a first aspect, the present invention also provides a fermentation process for the production of a cellulase enzyme mixture comprising (a) providing a fungal cell of the genus Myceliophthora or a taxonomically equivalent genus; (b) culturing the fungal cell in a submerged liquid batch culture a carbon source, wherein the carbon source is not cellulose; (c) culturing the fungal cell from the batch culture from step (b) in a submerged liquid fed-batch, continuous, or combined fed-batch and continuous culture; and (d) providing the culture of step (c) with a feed solution having a carbon source, wherein about 100 wt % of the carbon source is a non-inducing carbon source, the feed solution being provided at a rate that maintains the concentration of the non-inducing carbon source in the culture below that which would otherwise repress production of the cellulase enzyme mixture.

In another aspect, the present invention also provides a fermentation process for the production of a cellulase enzyme mixture comprising (a) providing a fungal cell of the genus Myceliophthora or a taxonomically equivalent genus; (b) culturing the fungal cell in a submerged liquid batch culture comprising a carbon source that is not a cellulase-inducing carbon source, (c) culturing the fungal cell from the batch culture from step (b) in a submerged liquid fed-batch, continuous, or combined fed-batch and continuous culture; and (d) providing the culture of step (c) with a feed solution having a carbon source, wherein about 100 wt % of the carbon source is a non-inducing carbon source, at a rate that maintains the concentration of the non-inducing carbon source in the culture below that which would otherwise repress production of the cellulase enzyme mixture.

Such fermentation process produces a culture filtrate containing the cellulase enzyme mixture at a concentration of at least 10 g protein per litre of filtrate. In some embodiments, the fermentation process produces a culture filtrate containing the cellulase enzyme mixture at a concentration of at least 25 g protein per litre of filtrate

In one embodiment, the non-inducing carbon source in the feed solution of step (d) is selected from the group consisting of glucose, dextrose, sucrose, molasses, fructose, glycerol, xylose, and any combination thereof.

In another embodiment, the feed solution is provided at a rate that maintains the concentration of the non-inducing carbon source in the culture below 2 g/L.

The present invention also provides the fermentation process as described above, wherein the step of culturing in step (c) is a submerged liquid fed-batch culture provided with the feed solution at a rate of from about 0.2 to about 4 g carbon/L of culture/h and the culture filtrate contains the cellulase mixture at a concentration of least 25 g protein/L filtrate.

The present invention also provides the fermentation process as described above, wherein the step of culturing in step (c) is a submerged liquid continuous culture provided with the feed solution at a dilution rate of from about 0.001 to about 0.1 h⁻¹ and the culture filtrate contains the cellulase mixture at a concentration of least 10 g protein/L filtrate.

The present invention provides the fermentation process as defined above, wherein the fungal cell is a strain of Myceliophthora thermophila, Thielavia heterothallica, Sporotrichum thermophile, Chrysosporium thermophile, Chrysosporium lucknowense or Corynascus heterothallicus.

In some embodiments, the fungal cell may comprise one or more mutations that result in production of a cellulase enzyme mixture in the absence of cellulase-inducing carbon source(s) (for example, cellulose, cellobiose, lactose, sophorose, or gentiobiose) and/or one or more mutations that result in production of a cellulase enzyme mixture in the presence of a non-inducing carbon source (for example, glucose, sucrose, xylose, fructose, molasses or glycerol).

In other embodiments, the fungal cell may be genetically modified to enhance or reduce the expression of one or more protein of interest, including but not limited to cellulase, hemicellulase, beta-glucosidase, esterase, cellulase-enhancing polypeptide, cellobiose dehydrogenase, laccase, lignin peroxidase, manganese peroxidase, beta-glucanase, protease, amylase, and glucoamylase. The one or more protein of interest may be homologous or heterologous with respect to the fungal cell.

The present invention is also directed to a method of hydrolyzing a cellulosic substrate with the cellulase enzyme mixture produced by the fermentation process as described above. The cellulosic substrate may be a pretreated lignocellulosic substrate.

The present invention is in part based on the surprising discovery that carbon sources consisting substantially of glucose, dextrose, glycerol, sucrose, fructose, xylose, or any combination thereof, can be used to produce high levels of cellulase protein when used as a feed for fed-batch or continuous submerged liquid culture fermentations of cellulase-producing fungal cells of the genus Myceliophthora. The protein concentration of the resulting culture filtrates are similar to that found in culture filtrates reported for batch and fed-batch cultures of cellulase-producing Myceliophthora from batch or fed-batch cultures in which the carbon source consists of one or more a cellulase-inducing carbon source such as cellulose, cellobiose, or lactose.

### BRIEF DESCRIPTION OF THE DRAWINGS

These and other features of the invention will become more apparent from the following description in which reference is made to the appended drawings wherein:

FIG. 1 shows the production of a cellulase mixture by Myceliophthora thermophila in (A) 5 L fed-batch or (B) 14 L submerged fed-batch liquid culture fermentation using 100% glucose as the carbon source.

FIG. 2 shows the production of a cellulase mixture by Myceliophthora thermophila in a 5 L fed-batch submerged liquid culture fermentation using a mixture of glucose and cellulase-inducing disaccharides as carbon source.

FIG. 3 shows the production of a cellulase mixture by Myceliophthora thermophila in a 14 L fed-batch submerged liquid culture fermentation using 100% glycerol as the carbon source.

FIG. 4 shows the production of a cellulase mixture by Myceliophthora thermophila in a 5 L fed-batch submerged fed-batch liquid culture fermentation using (A) 100% xylose or (B) a mixture of 90:10 (w:w) glucose:xylose as the carbon source.

FIG. 5 shows the production of a cellulase mixture by Myceliophthora thermophila in a 14 L fed-batch submerged fed-batch liquid culture fermentation using 100% sucrose as the carbon source.

FIG. 6 shows the production of a cellulase mixture by Myceliophthora thermophila in a 14 L fed-batch submerged liquid culture fermentation using 100% molasses as the carbon source.

FIG. 7 shows the production of a cellulase mixture by Myceliophthora thermophila in a 14 L fed-batch submerged fed-batch liquid culture fermentation using (A) 100% fructose or (B) a mixture of 50:50 (w:w) glucose:fructose as the carbon source.

### DETAILED DESCRIPTION

The present invention provides a production of cellulase from fermentation of fungal cells, preferably in submerged liquid culture fermentations.

The following description is of an embodiment by way of example only and without limitation to the combination of features necessary for carrying the invention into effect. The headings provided are not meant to be limiting of the various embodiments of the invention. Terms such as “comprises,” “comprising,” “comprise,” “includes,” “including,” and “include” are not meant to be limiting. In addition, the use of the singular includes the plural, and “or” means “and/or” unless otherwise stated. Unless otherwise defined herein, all technical and scientific terms used herein have the same meaning as commonly understood by one of ordinary skill in the art.

### Cellulase Enzyme Mixture

The following definitions refer to classification of cellobiohydrolases, endoglucanases, beta-glucosidases, hemicellulases and related proteins as defined by the by the Joint Commission on Biochemical Nomenclature of the International Union of Biochemistry and Molecular Biology (Published in Enzyme Nomenclature 1992, Academic Press, San Diego, Calif., ISBN 0-12-227164-5; with supplements in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650, each of which are incorporated herein by reference; also see: chem.qmul.ac.uk/iubmb/enzyme/) and to the glycoside hydrolase (GH) families as defined by the CAZy system which is accepted as a standard nomenclature for glycohydrolase enzymes (Coutinho, P. M. & Henrissat, B., 1999, “Carbon-active enzymes: an integrated database approach.” In Recent Advances in Carbon Bioengineering, H. J. Gilbert, G. Davies, B. Henrissat and B. Svensson eds., The Royal Society of Chemistry, Cambridge, pp. 3-12, which is incorporated herein by reference; also see: afmb.cnrs-mrs.fr/CAZY/) and is familiar to those skilled in the art.

A cellulase enzyme mixture, as defined herein, is an enzyme composition comprising one or more cellulase or cellulose-degrading enzymes. The term cellulase (or cellulase enzymes) broadly refers to enzymes that catalyze the hydrolysis of the β-1,4-glucosidic bonds joining individual glucose units in the cellulose polymer. The catalytic mechanism involves the synergistic actions of endoglucanases (E.C. 3.2.1.4) and cellobiohydrolases (E.C. 3.2.1.91). Endoglucanases hydrolyze accessible glucosidic bonds in the middle of the cellulose chain, while cellobiohydrolases release cellobiose from these chain ends processively. Cellobiohydrolases are also referred to as exoglucanases. Most cellulases have a similar modular structure, which consists of one or more catalytic domain and one or more carbohydrate-binding modules (CBM) joined by flexible linker peptides. Most cellulases comprise at least one catalytic domain of GH Family 5, 6, 7, 8, 9, 12, 44, 45, 48, 51, 61 and 74.

Genome sequencing of a cellulase-producing Myceliophthora thermophila strain (ATCC No. 42464, formerly classified as Sporotrichum thermophile, see URL: genome.jgi-psf.org/Spoth2/Spoth2.info.html) reveals the presence of at least six genes encoding cellobiohydrolases (including four GH 7 cellobiohydrolases, including CBH1a and CBH1b and two GH6 cellobiohydrolases, CBH2a and CBH2b), and at least four genes encoding endoglucanases representing GH Families 5 and 7.

The cellulase enzyme mixture produced by the fermentation process of the present invention may include one or more cellulase-enhancing proteins. A cellulase-enhancing protein is a protein that enhances the rate or extent of cellulose hydrolysis by cellulase enzymes but does not exhibit significant cellulose-degrading activity on its own. Cellulase-enhancing proteins include, but are not limited to, proteins classified in GH Family 61, swollenins and expansins. Genome sequencing of Myceliophthora thermophila strain ATCC No. 42464 reveals the presence of at least 20 genes predicted to encode cellulase-enhancing proteins of GH Family 61.

The cellulase enzyme mixture produced by the fermentation process of the present invention may include one or more hemicellulases or hemicellulose degrading enzymes—i.e., enzyme capable of hydrolysing the glycosidic bonds in a hemicellulose polymer. Hemicellulases include, but are not limited to, xylanase (E. C. 3.2.1.8), beta-mannanase (E.C. 3.2.1.78), alpha-arabinofuranosidase (E.C. 3.2.1.55), beta-xylosidases (E.C. 3.2.1.37), and beta-mannosidase (E.C. 3.2.1.25). Hemicellulases typically comprise a catalytic domain of Glycoside Hydrolase Family 5, 8, 10, 11, 26, 43, 51, 54, 62 or 113. Genome sequencing of Myceliophthora thermophila strain ATCC No. 42464 reveals the presence of at least ten genes encoding xylanases, at least three genes encoding mannanases, and at least two genes encoding alpha-arabinofuranosidases.

The cellulase enzyme mixture produced by the fermentation process of the present invention may include one or more beta-glucosidases (E.C. 3.2.1.21), which hydrolyze cellobiose to glucose. Beta-glucosidases typically comprise catalytic domains of GH Family 1 or 3 but usually do not comprise a CBM. Genome sequencing of Myceliophthora thermophila strain ATCC No. 42464 reveals the presence of at least eight genes encoding beta-glucosidases.

The cellulase enzyme mixture produced by the fermentation process of the present invention may include one or more lignin degrading enzymes, including but not limited to laccases (E.C. 1.10.3.2), lignin peroxidases (E.C. 1.11.1.14), manganese peroxidases (E.C. 1.11.1.13) and cellobiose dehydrogenases (E.C. 1.1.99.18). Genome sequencing of Myceliophthora thermophila strain ATCC No. 42464 reveals the presence of at least four genes encoding lignin-degrading enzymes.

The cellulase enzyme mixture produced by the fermentation process of the present invention may include one or more esterases, including but not limited to acetyl xylan esterases (E.C. 3.1.1.72) and ferulic acid esterases (E.C. 3.1.1.73). Genome sequencing Myceliophthora thermophila strain ATCC No. 42464 reveals the presence of at least four genes encoding acetyl xylan esterases and ferulic acid esterases.

The cellulase enzyme mixtures produced by the fermentation process of the present invention may include one or more additional enzyme activities known to be produced and secreted by Myceliophthora and its taxonomic equivalents including pectinases, pectate lyases, galactanases, amylases, glucoamylases, glucuronidases and galacturonidases.

The practice of the fermentation process of the present invention is not limited by the particular composition of the cellulase enzyme mixture. However, depending on the intended use of the cellulase enzyme mixture produced, it may be desirable that cellulases comprise from about 20 wt % to about 100 wt %, for example about 20, 30, 40, 50, 60, 70, 80, 90 or 100 wt %, of the proteins present in the cellulase enzyme mixture.

### Fungal Cell

In the fermentation process of the present invention, the fungal cell is a species of Myceliophthora, including anamorphs and teleomorphs thereof, as well as recognized synonymous genera such as Sporotrichum, Thielavia, Corynascus, Chrysosporium or Ctenomyces. For example, the following species are anamorphs or teleomorphs and may therefore be considered as synonymous: Myceliophthora thermophila, Sporotrichum thermophile, Sporotrichum thermophilum, Sporotrichum cellulophilum, Chrysosporium thermophile, Corynascus heterothallicus, and Thielavia heterothallica. It will be understood that for the aforementioned species, the fungal cell presented herein encompasses both the perfect and imperfect states, and other taxonomic equivalents, e.g., anamorphs, regardless of the species name by which they are known. Further examples of taxonomic equivalents can be found, for example, in Cannon, Mycopathologia 111:75-83, 1990; Moustafa et al., Persoonia 14:173-175, 1990; Stalpers, Stud. Mycol. 24, 1984; Upadhyay et al., Mycopathologia 87:71-80, 1984; Guarro et al., Mycotaxon 23: 419-427, 1985; Awao et al., Mycotaxon 16:436-440, 1983; von Klopotek, Arch. Microbiol. 98:365-369, 1974; and Long et al., 1994, ATCC Names of Industrial Fungi, ATCC, Rockville Md. Those skilled in the art will readily recognize the identity of appropriate equivalents. Accordingly, it will be understood that, unless otherwise stated, the use of a particular genus and/or species designation in the present disclosure also refers to genera and species that are related by anamorphic or teleomorphic relationship, as well as genera and species that have been or may be reclassified into one of the claims genera or species in the future.

The fungal cell used in the fermentation process of the present invention is capable of producing one or more cellulase enzyme. By “capable of producing”, it is meant that the fungal cell comprises one or more genes encoding cellulase enzymes.

The fungal cell used in the fermentation process of the present invention may contain one or more mutations that result in increased production of one or more cellulase proteins, relative to a fungal cell lacking such mutations, in the absence of a cellulase-inducing carbon source. For the purposes herein, a cellulase-inducing carbon source, or CIC, is any monosaccharide, disaccharide, oligosaccharide or polysaccharide that, when provided to the fungal cell as a carbon source, activates the production of cellulase activity, and includes breakdown products of cellulose such as soluble cellodextrins. Commonly used CIC include, but are not limited to, cellulose (including pure cellulose as well as lignocellulose or other cellulose-containing biomass), cellulose derivatives (for example, carboxymethyl cellulose), cellobiose, sophorose, gentiobiose, lactose, and any combination thereof.

The fungal cell used in the fermentation process of the present invention may contain one or more mutations that result in increased production of one or more cellulase proteins in the presence of a non-inducing carbon source relative to a fungal cell lacking such mutations. For the purposes herein, a non-inducing carbon source, or NIC, is any mono-, di-, or oligosaccharide that, when provided to the fungal cell as a carbon source, either alone or in the presence of a CIC, represses the production of cellulase activity. NIC includes, but is not limited to, glucose, sucrose, glycerol, dextrose, molasses, fructose, xylose, and any combination thereof.

As used herein, a “mutation” includes, but is not limited to, (a) heritable changes to the sequence or structure of the fungal cell's genomic DNA resulting, for example, from random mutagenesis and selection, adaptation, or epigenetic changes and (b) genetic modification to introduce polynucleotide sequences into the fungal cell using recombinant means.

As used herein, random mutagenesis and selection refers to the process of creating by natural or artificial means, including subjecting the fungal cell to irradiation or chemical mutagenesis, a library of mutated strains, which are then screened for a desired altered phenotype. Adaptation, also referred to as “adaptive evolution” or “evolutionary engineering”, refers to any method or procedure employed to influence the phenotype and genetic profile of a fungal cell through the use of exposure to environmental challenges, and subsequent selection of a modified fungal cell with the desired altered phenotype. “Epigenetic changes” are defined as heritable changes in chromatin structure that alter the expression of one or more genes in an organism, including but not limited to, histone methylation, histone acetylation, ubiquitination, phosphorylation or sumoylation, and DNA methylation.

As used herein, “recombinant means”, “recombinant technology”, “genetic modification”, or “genetically modified” refers to any of several well-known techniques for the direct manipulation of an organism's genome. For example, gene knockout (insertion of an inoperative polynucleotide sequence, often replacing the endogenous operative sequence, into an organism's chromosome), gene knock-in (insertion of a protein-coding polynucleotide sequence into an organism's chromosome), and gene knockdown (insertion of polynucleotide sequences that encode antisense RNA or small interfering RNA, i.e., RNA interference (RNAi)) techniques are well known in the art. Methods for decreasing the expression of a gene also include partial or complete deletion of the gene, and disruption or replacement of the promoter of the gene such that transcription of the gene is greatly reduced or even inhibited. For example, the promoter of the gene can be replaced with a weak promoter, as exemplified by U.S. Pat. No. 6,933,133, which is incorporated by reference herein in its entirety.

By increased production, it is meant that a culture filtrate from the fungal cell containing the one or more mutations has measurably more cellulase activity or a higher concentration of protein than a culture filtrate from a fungal cell lacking such mutations when grown under essentially identical conditions of medium composition, time, cell density, temperature, and pH.

There are several assays for measuring cellulase activity known to one of skill in the art. It should be understood, however, that the practice of the present invention is not limited by the method used to assess cellulase activity. Methods to measure cellulase activity are published (e.g., Methods in Enzymology 160, Biomass Part A: Cellulose and Hemicellulose, Wood, W. A. and Kellogg, S. T., eds, Academic Press Inc. 1988; Ghose, T. K. (1987) Pure & Appl. Chem. 59(2):257-268) and include, for example, release of glucose or soluble oligosaccharides from a cellulose substrate, release of a chromophore or fluorophore from a cellulose derivative, e.g., azo-CMC, or from a small, soluble substrate such as methylumbelliferyl-beta-D-cellobioside, para-nitrophenyl-beta-D-cellobioside, para-nitrophenyl-beta-D-lactoside and the like. For example, hydrolysis of cellulose can be monitored by measuring the enzyme-dependent release of reducing sugars, which are quantified in subsequent chemical or chemienzymatic assays known to one of skill in the art, including reaction with dinitrosalisylic acid (DNS). In addition, cellulose or colorimetric substrates (cellulose derivatives or soluble substrates) may be incorporated into agar-medium on which a host microbe expressing and secreting one or more cellulase enzymes is grown. In such an agar-plate assay, activity of the cellulase is detected as a colored or colorless halo around the individual microbial colony expressing and secreting an active cellulase.

Methods to measure protein concentration include the methods of Bradford (Bradford, M. M. et al. (1976) Anal. Biochem. 72: 248-254), Lowry (Lowry O H, et al. (1951) J. Biol. Chem. 193: 265-175), and Smith (Smith, P. K., et al. (1985). Anal. Biochem. 150, 76-85). Increased production of individual cellulase enzymes can be measured, for example, with immunochemical methods such as ELISA (Van Weemen, B. K. et al. (1971) FEBS Letters 15: 232-236) with antibodies specific to the individual enzyme.

In some embodiments, the fungal cell used in the fermentation process of the present invention is genetically modified to increase or decrease the expression or activity of one or more protein of interest. The protein of interest may be a cellulase, a hemicellulase, a beta-glucosidase, an esterase, a cellulase-enhancing polypeptide, a cellobiose dehydrogenase, a laccase, a lignin peroxidase, a manganese peroxidase, a beta-glucanase, a protease, an amylase, or a glucoamylase. The protein of interest may be secreted from the fungal cell. The protein of interest may be homologous or heterologous with respect to the fungal cell. For the purposes herein, a homologous protein of interest is encoded by a polynucleotide sequence that naturally occurs in, or is isolated or derived from, the same or taxonomically equivalent taxonomic species as the fungal cell. Furthermore, as is recognized by one of skill in the art, a homologous protein may contain one or more insertions, deletions and substitutions and still be considered to be “derived from” a given species. Such one or more insertions, deletions and substitutions may result in the increase or decrease in the expression or activity of the protein of interest. A heterologous protein of interest is encoded by a polynucleotide sequence that naturally occurs in, or is isolated or derived from, a different taxonomic species from the fungal cell.

As used herein, in respect of polynucleotide sequences, “derived from” refers to the isolation of a target polynucleotide sequence using one or more molecular biology techniques known to those of skill in the art including, but not limited to, cloning, sub-cloning, amplification by PCR, in vitro synthesis, and the like. Furthermore, as is recognized by one of skill in the art, a polynucleotide sequence that is derived from a target polynucleotide sequence may be modified by one or more insertions, deletions and substitutions and still be considered to be “derived from” that target nucleotide sequence. Such one or more insertions, deletions and substitutions may result in the increase or decrease in the expression or activity of the protein of interest encoded by the polynucleotide sequence and may be located within a promoter sequence, the 5′ or 3′ untranslated regions, or within the coding region for the protein of interest.

As used herein with respect to polynucleotide sequences, “isolated” or “isolation” means altered from its natural state by virtue of separating the nucleic acid sequence from some or all of the naturally-occurring nucleic acid sequences with which it is associated in nature.

In some embodiments, the fungal cell may be genetically modified to at least partially delete one or more gene(s). As used herein, a gene deletion or deletion mutation is a mutation in which part of a sequence of the DNA making up the gene is missing. Thus, a deletion is a loss or replacement of genetic material resulting in a complete or partial disruption of the sequence of the DNA making up the gene. Any number of nucleotides can be deleted, from a single base to an entire piece of a chromosome. In some embodiments, complete or near-complete deletion of the gene sequence is contemplated.

In other embodiments, the fungal cell may be genetically modified by transformation of the fungal cell with a genetic construct. As used herein, “genetic construct” refers to an isolated polynucleotide comprising elements necessary for increasing or decreasing the expression of a protein of interest. These elements may include, but are not limited to, a coding region comprising a polynucleotide sequence that encodes a protein product, a promoter operably linked to the coding region and comprising a polynucleotide sequence that directs the transcription of the coding region, and a sequence encoding a secretion signal peptide and operably linked to the coding region, or targeting polynucleotide sequences that direct homologous recombination of the construct into the genome of the fungal cell.

The terms “secretion signal peptide”, “secretion signal” and “signal peptide” refer to any sequence amino acids which participate in the secretion of the mature or precursor forms of a secreted protein into the extracellular culture medium. The signal sequence may be endogenous or exogenous with respect to the fungal cell. The signal sequence may be that normally associated with the protein of interest, from a gene encoding another secreted protein, be a “hybrid signal sequence” containing partial sequences from two or more genes encoding secreted proteins.

As understood by one of ordinary skill in the art, the coding region, promoter, and sequence encoding a secretion signal peptide may be derived from the fungal cell or from a different organism, and/or be synthesized in vitro. For example, the promoter and sequence encoding a secretion signal peptide may be derived from one or more genes encoding proteins that are highly expressed and secreted when the fungal cell is grown in the fermentation process defined below, such as gene encoding a cellulase, beta-glucosidase, cellulase-enhancing protein, a hemicellulase or any combination thereof. However, it should be understood that the practice of the present invention is not limited by the choice of promoter or sequence encoding a secretion signal peptide in the genetic constructs.

These polynucleotide elements may also be altered or engineered by replacement, substitution, addition, or elimination of one or more nucleic acids relative to a naturally-occurring polynucleotide. The practice of this invention is not constrained by such alterations to the elements comprising the genetic construct

A genetic construct may contain a selectable marker for determining transformation of a host cell. The selectable marker may be present on the genetic construct or the selectable marker may be a separate isolated polynucleotide that is co-transformed with the genetic construct. Choices of selectable markers are well known to those skilled in the art and include genes (synthetic or natural) that confer to the transformed fungal cells the ability to utilize a metabolite that is not normally metabolized by the microbe (e.g., the A. nidulans amdS gene encoding acetamidase and conferring the ability to grow on acetamide as the sole nitrogen source) or antibiotic resistance (e.g., the Escherichia coli hph gene encoding hygromycin-beta-phosphotransferase and conferring resistance to hygromycin). If the host fungal cell expresses little or none of the chosen marker activity, then the corresponding gene may be used as a marker. Examples of such markers include trp, pyr4, pyrG, argB, leu, and the like. The corresponding host fungal cell would therefore lack functional gene corresponding to the marker chosen, e.g., a trp, pyr, arg, or leu gene.

A genetic construct may contain a transcriptional terminator that is functional in the fungal cell, as would be known to one of skill in the art. The transcriptional terminator may be positioned immediately downstream of a coding region. The practice of the invention is not constrained by the choice of transcriptional terminator that is sufficient to direct the termination of transcription in the host fungal cell.

A genetic construct may contain additional polynucleotide sequences between the various sequence elements as described herein. These sequences, which may be natural or synthetic, may result in the addition of one or more of the amino acids to the protein encoded by the construct. The practice of the invention is not constrained by the presence of additional polynucleotide sequences between the various sequence elements of the genetic constructs present in the fungal cell.

Methods of introducing a genetic construct into a fungal cell are familiar to those skilled in the art and include, but are not limited to, calcium chloride treatment of fungal protoplasts to weaken the cell membranes, addition of polyethylene glycol to allow for fusion of cell membranes, depolarization of cell membranes by electroporation, or shooting the construct through the cell wall and membranes via microprojectile bombardment with a particle gun. The practice of the present invention is not constrained by the method of introducing the genetic constructs into the fungal cell.

For the purposes described herein, the term “increased expression” means at least about a 20% increase in the level of transcript for a given gene, or at least a 20% increase in the expression or activity of the protein encoded by a given gene, in a modified fungal cell as compared to that of the same gene in a parental fungal cell, when grown under identical or nearly identical conditions of medium composition, temperature, pH, cell density and age of culture. For the purposes described herein, the term “decreased expression” means at least about a 20% decrease in the level of transcript for a given gene, or at least a 20% decrease in the expression or activity of the protein encoded by a given gene, in a modified fungal cell as compared to that of the same gene in a parental fungal cell when grown under identical or nearly identical conditions of medium composition, temperature, pH, cell density and age of culture.

### Fermentation Process

The production of cellulase enzymes by fungi such as Myceliophthora is typically regulated by the available carbon source. In the context of the fermentation process of the present invention, a carbon source is a carbohydrate that can be utilized by the fungal cell to produce energy. As used herein, organic acids are not considered as carbon sources. Similarly, organic nitrogen compounds such as urea, amino acids, peptides, proteins, in pure or raw form (e.g., corn steep liquor) are not considered as carbon sources.

Typically, fungal cells produce high levels of cellulase enzymes when the available carbon source is a cellulase-inducing carbon source (CIC) such as cellulose, cellulose derivatives, or beta-linked oligosaccharides or disaccharides such as cellobiose, sophorose, gentiobiose or lactose, and not produced when the carbon source consists of one or more one or more cellulase-repressing or non-inducing carbon source(s).

As used herein, the terms non-inducing carbon source (NIC), and cellulase-repressing carbon source may be used synonymously and include those carbohydrates and other non-carbohydrate carbon sources (e.g., glycerol, sugar alcohols and organic acids), that can be readily metabolized by, but that are known either to not induce or to repress the production of cellulase from, Myceliophthora and taxonomically equivalent genera. Typically, NIC, when provided alone or in combination with CIC, results in the production of negligible or very low amounts of cellulase enzyme. For the purposes herein, NIC includes, but is not limited to, glucose, dextrose, sucrose, xylose, fructose glycerol, and combinations thereof, whether in pure form or in semi-purified form, such as molasses.

Cellulase enzyme mixtures are typically produced by subjecting an actively growing fungal culture to media (solid or liquid) containing a cellulase-inducing carbon source, or CIC (e.g., cellulose, cellobiose, sophorose, gentiobiose, lactose or combinations thereof), as well as other nutrients required for cell growth, at temperatures and pH suitable for the host cell. An actively growing fungal culture may be prepared by inoculating an initial growth medium with spores or mycelia and growing the culture for a period of one to several days at a temperature optimal for growth of the fungal cells, for example, from about 20° C. to about 50° C. As is known to one of skill in the art, the initial growth medium may be solid or liquid and may be a defined mineral medium or a rich medium that typically, though not necessarily, contains glucose or other non-inducing or cellulase-repressing carbon source (e.g., glycerol, fructose or sucrose) as the carbon source. The actively growing culture may be prepared in the same or different vessel or reactor as that used for the fermentation process of the present invention.

The fermentation process of the present invention comprises culturing a fungal cell of the genus Myceliophthora (or its taxonomic equivalents defined herein) in a submerged liquid culture. A submerged liquid culture, as defined herein, is a microbial culture in which the microbial cells are suspended in a liquid medium containing nutrients required for maintaining the viability of the cells and which is agitated at a sufficient rate to ensure distribution of the cells throughout the medium and to prevent formation of concentration gradients of nutrients. For example, the culture may be agitated by shaking from about 100 to about 1000 rpm, or any rate therebetween, or by impeller stirring with a tip speed of from about 0.5 to about 10 m/s, or any rate therebetween, for example from about 0.5 to about 3 m/s. An alternative parameter to measure agitation that is known to one of skill in the art, particularly as it relates to agitation in bioreactors, is horsepower (hp) per 100 gallons. In the fermentation process of the present invention, the submerged liquid culture may be agitated at from about 0.2 hp/100 gallons to about 15 hp/100 gallons.

In the fermentation process of the present invention, the fungal cell is first cultured in a batch fermentation in which the carbon source is not a cellulase-inducing carbon source. In a batch process, all the necessary materials, with the exception of oxygen for aerobic processes, are placed in a reactor at the start of the operation and the fermentation is allowed to proceed until the carbon source is depleted. The batch fermentation of the present invention may be carried out in a shake-flask or a bioreactor.

Upon completion of the batch phase of the fermentation process, which is typically identified by the depletion of essentially all of the available carbon source, for example, when the concentration of all carbon sources in the culture filtrate is no more than 1 g/L, the fungal cell is cultured in a fed-batch, continuous or combined fed-batch and continuous submerged liquid culture. As used herein, a fed-batch culture is one that is fed continuously, intermittently, or sequentially with a feed containing the carbon source and optionally, one or more media components, without the removal of the culture fluid. A continuous culture is one which is fed continuously or intermittently with a feed containing the carbon source and optionally, one or more media components, and from which culture fluid is removed continuously or intermittently at volumetrically equal rates to maintain the culture at a steady growth rate. Continuous fermentation process may also be referred to as CSTR (continuous stirred-tank reactor) fermentations. One example of a continuous fermentation process is a chemostat, in which the growth rate of the microorganism is controlled by the supply of one limiting nutrient in the medium.

Fed-batch and continuous processes are typically carried out in one or more bioreactors. Typical bioreactors used for microbial fermentation processes include, but are not limited to, mechanically agitated vessels or those with other means of agitation (such as air injection). Bioreactors may be temperature and pH-controlled. Usually there are means provided to clean the reactor, sometimes in place. Means may also be provided to sanitize or sterilize the bioreactor prior to introduction of the target organism so as to minimize or prevent competition for carbon sources from other organisms. Bioreactors may be constructed from many materials, but most often are of glass or stainless steel. Provisions are generally made for sampling (in a manner that prevents or minimizes the introduction of undesirable competing organisms). Means to obtain other measurements are often provided (e.g., ports and probes to measure dissolved oxygen concentration or concentration of other solutes such as ammonium ions). The practice of the invention is not limited by the choice of bioreactor(s).

In the fermentation process of the present invention, the fed-batch, continuous or combined fed-batch and continuous submerged liquid culture is provided with a feed solution in which substantially all of the carbon source is a non-inducing carbon source. For example, the carbon source may be about 100 wt % non-inducing carbon source (NIC). In the context of the feed solution provided to the fed-batch, continuous or combined fed-batch and continuous submerged liquid culture, “about 100 wt % non-inducing carbon source” means that the non-inducing carbon source contributes more than 99 wt % of the total combined weight of all non-inducing carbon sources and all cellulase-inducing carbon sources in the feed solution. When a mixture of two or more NIC is used in the feed solution, the total weight of all of the NIC is more than 99 wt % of total combined weight of all NIC and all CIC in the feed solution.

In addition to a carbon source, the initial medium used for the batch phase, as well as the feed solution provided to the fed-batch and/or continuous submerged liquid culture, may contain one or more additional components, vitamins, minerals and salts required for growth of the fungal cell as in known to one of skill in the art. Nitrogen sources may be inorganic and/or organic in nature and include, but is not limited to, one or more amino acids, any number of protein hydrolysates (peptone, tryptone, casamino acids), yeast extract, corn-steel liquor, ammonia, ammonium hydroxide, ammonium salts, urea, nitrate and combinations thereof. The practice of the fermentation process of the present invention is not limited by the additional components of the feed solution.

The feed solution is provided to the fermentation process at a rate, the feed rate, which maintains the concentration of NIC in the culture medium below that which would otherwise repress the production of the cellulase enzyme mixture. For example, the concentration of NIC in the culture medium may be maintained below about 2 g/L, for example, below 2.0, 1.8, 1.6, 1.4, 1.2, 1.0, 0.8, 0.6, 0.4, 0.2, 0.1, 0.05, 0.01, and 0 g/L, or any concentration therebetween.

In the fermentation process of the present invention, the feed solution may be provided to a fed-batch culture at a feed rate of from about 0.2 to about 4 g carbon/L culture/h (or from about 0.5 to about 10 g carbohydrate/L culture/h), for example 0.2, 0.3, 0.4, 0.6, 0.8, 1.0, 1.2, 1.4, 1.6, 1.8, 2.0, 2.2, 2.5, 3.0, 3.5, and 4.0 g carbon/L culture/h or any rate therebetween. Alternatively, the feed solution may be provided to a continuous culture at a dilution rate of from about 0.001 to 0.1 h⁻¹, or any dilution rate therebetween, for example at about 0.001, 0.005, 0.01, 0.02, 0.03, 0.04, 0.05, 0.06, 0.07, 0.08, 0.09, 0.1 h⁻¹, or any dilution rate therebetween.

The process of the present invention may be carried out at a temperature from about 20° C. to about 50° C., or any temperature therebetween, for example from about 30° C. to about 45° C., or any temperature therebetween, or from 20, 22, 25, 28, 30, 32, 35, 38, 40, 42, 45, 48, 50° C., or any temperature therebetween.

The process of the present invention may be carried out at a pH from about 3.0 to 8.0, or any pH therebetween, for example from about pH 3.5 to pH 7.0, or any pH therebetween, for example from about pH 3.0, 3.2, 3.5, 3.8, 4.0, 4.2, 4.5, 4.8, 5.0, 5.2, 5.4, 5.5, 5.7, 5.8, 6.0, 6.2, 6.5, 6.8, 7.0, 7.2, 7.5, 7.8, 8.0 or any pH therebetween. The pH may be controlled by the addition of a base, such as ammonium or sodium hydroxide, or by the addition of an acid, such as phosphoric acid.

The process of the present invention may be carried out over a period of about 1-90 days, or any period therebetween, for example between 3 and 30 days, or any amount therebetween, between 3 and 8 days, or any amount therebetween, or from 1, 2, 3, 4, 5, 6, 7, 8, 9, 10, 12, 14, 16, 18, 20, 22, 24, 26, 28, 30, 40, 50, 60, 70, 80, or 90 days, or any amount therebetween. In some embodiments, the fermentation process of the present invention is carried out over a period of 72 to 196 hours, for example, 72, 96, 120, 144, 168, or 196 hours, an any number of hours therebetween.

The process of the present invention may be performed in cultures having a volume of at least 0.5 litre, for example from about 0.5 to about 1,000,000 litres, or any amount therebetween, for example, 5 to about 400,000 litres, or any amount therebetween, 10 to about 200,000 litres, or any amount therebetween, or 2,000 to about 200,000 litres, or any amount therebetween, or from about 0.5, 1, 10, 50, 100, 200, 400, 600, 800, 1000, 2000, 4000, 6000, 8000 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 150,000, 200,000, 300,000, 400,000, 500,000, 750,000 or 1,000,000 litres in volume, or any amount therebetween.

The fermentation process of the present invention may be performed aerobically, in the presence of oxygen, or anaerobically, in the absence of oxygen. For example, the process may be performed aerobically such that air or oxygen gas is provided to the submerged liquid culture at a superficial gas velocity of from about 0.001 to about 100 cm/s, or any rate therebetween, for example any rate from about 0.01 to about 20 cm/s, or any rate therebetween. An alternative parameter to measure aeration rate that is known to one of skill in the art is vessel volumes per minute (vvm). In the fermentation process of the present invention, air or oxygen gas is provided to the submerged liquid culture at a rate of from about 0.5 to about 5 vvm, or any rate therebetween. Antifoaming agents (either silicone, or non-silicone based) may be added to control excessive foaming during the process as required and as is known to one of skill in the art.

A fed-batch fermentation process of the present invention may produce at least 25 g/L of secreted protein. For example the fed-batch fermentation process described herein may produce from about 25 to about 200 g/L secreted protein, or any amount therebetween, for example about 25, 30, 35, 40, 45, 50, 55, 60, 65, 70, 75, 80, 85, 90, 100, 110, 120, 130, 140, 150, 160, 170, 180, 190, 200 g/L of secreted protein, or any amount therebetween.

A continuous fermentation process of the present invention may produce at least 10 g/L of secreted protein. For example the fed-batch fermentation process described herein may produce from about 10 to about 100 g/L secreted protein, or any amount therebetween, for example about 10, 15, 20, 25, 30, 35, 40, 45, 50, 55, 60, 65, 70, 75, 80, 85, 90, 100 g/L of secreted protein, or any amount therebetween.

The production of cellulase mixtures in fed-batch fermentations of M. thermophila in fed-batch fermentation processes of the present invention is shown in Table 1. Fermentations in which the carbon source consisted of glucose, sucrose, molasses, fructose, xylose, a mixture of 90:10 (w:w) glucose: xylose, and a mixture of 50:50 (w:w) glucose: fructose, all produced culture filtrates containing similar concentration of total protein with similar cellulase activities to that produced by control fermentation processes in which the carbon source consisted of cellulose and glucose or glucose and inducing disaccharides. Although the fermentation using glycerol as a carbon source produced a cellulase mixture containing less, but still significantly high amounts of total protein, the cellulase activity of the culture filtrate was similar to those of the other cellulase mixtures.

TABLE 1
Total protein concentration and cellulase activity of culture filtrates
produced in triplicate M. thermophila fed-batch fermentations

| | | Average Protein | Average Cellulase |
|---|---|---|---|
| | | Concentration | Activity |
| Carbon Source | Scale | (g/L) | (FPU/mg) |
| Glucose | 5 L | 47 ± 7 | 0.33 ± 0.02 |
| | 14 L | 59 ± 5 | 0.30 ± 0.04 |
| glucose + inducing | 5 L | 48 ± 6 | 0.37 ± 0.03 |
| disaccharides | | | |
| Glycerol | 14 L | 29 ± 2 | 0.27 ± 0.02 |
| Xylose | 5 L | 43 ± 3 | 0.55 ± 0.09 |
| 90:10 (w:w) | 5 L | 43 ± 3 | 0.54 ± 0.03 |
| glucose:xylose | | | |
| Sucrose | 14 L | 62 ± 4 | 0.26 ± 0.04 |
| Molasses | 14 L | 46 ± 4 | 0.33 ± 0.01 |
| fructose | 14 L | 72 ± 3 | ND* |
| 50:50 (w:w) | 14 L | 67 ± 1 | ND* |
| glucose:fructose | | | |
| Cellulose + glucose | 5 L | 61 ± 6 | 0.52 ± 0.01 |

*ND, not determined

Following fermentation, the fermentation broth containing the cellulase enzyme may be used directly, or the fungal cells can be removed, for example by filtration or centrifugation, to produce a culture filtrate. Low molecular solutes such as unconsumed components of the fermentation medium may be removed by ultrafiltration. The cellulase enzyme may be concentrated, for example, by evaporation, precipitation, sedimentation or filtration. Chemicals such as glycerol, sucrose, sorbitol and the like may be added to stabilize the cellulase enzyme. Other chemicals, such as sodium benzoate or potassium sorbate, may be added to the cellulase enzyme to prevent growth of microbial contaminants.

### Hydrolysis of Cellulosic Substrates

The cellulase enzyme mixture produced using the fermentation process of the present invention is useful for the hydrolysis of a cellulosic substrate. By the term “cellulosic substrate”, it is meant any substrate derived from plant biomass and comprising cellulose, including, but not limited to, pre-treated lignocellulosic feedstocks for the production of ethanol or other high value products, animal feeds, food products, forestry products, such as pulp, paper and wood chips, and textiles products.

### Hydrolysis of Pretreated Lignocellulose

The cellulase enzyme mixture produced using the fermentation process of the present invention may be used for the hydrolysis of a pretreated lignocellulosic substrate. A pretreated lignocellulosic substrate, or pretreated lignocellulose, is a material of plant origin that, prior to pretreatment, contains 20-90% cellulose (dry wt), more preferably about 30-90% cellulose, even more preferably 40-90% cellulose, for example 20, 22, 24, 26, 28, 30, 32, 34, 36, 38, 40, 42, 44, 46, 48, 50, 55, 60, 65, 70, 75, 80, 85, 90% or any % therebetween, and at least 10% lignin (dry wt), more typically at least 12% (dry wt) and that has been subjected to physical, chemical or biological processes to make the fiber more accessible and/or receptive to the actions of cellulolytic enzymes.

Methods for performing acid pretreatment of a lignocellulosic feedstock include the steam explosion process of U.S. Pat. No. 4,461,648, the continuous pretreatment processes described in U.S. Pat. No. 5,536,325; WO 2006/128304; and U.S. Pat. No. 4,237,226. Processes for pretreating lignocellulose with alkali include those described in U.S. Pat. Nos. 5,171,592; 5,037,663; 4,600,590; 6,106,888; 4,356,196; 5,939,544; 6,176,176; 5,037,663 and 5,171,592. Lignocellulosic feedstocks may also be subject to chemical treatment with organic solvents such as those described in U.S. Pat. No. 4,556,430 and U.S. Pat. No. 7,465,791. Pressurized water may also be a suitable pretreatment method for lignocellulosic feedstocks (see Weil et al. (1997) Appl. Biochem. Biotechnol. 68(1-2): 21-40).

The pretreated lignocellulosic feedstock may be processed after pretreatment by any of several steps, such as dilution with water, washing with water, buffering, filtration, or centrifugation, or a combination of these processes, prior to enzymatic hydrolysis, as is familiar to those skilled in the art. The pH of the pretreated feedstock slurry may be adjusted to a value that is amenable to the cellulase enzymes, which is typically between about 4 and about 8

The pretreated lignocellulose is subjected to enzymatic hydrolysis with the cellulase enzyme mixture produced by the fermentation process of the present invention. By the term “enzymatic hydrolysis”, it is meant a process by which cellulases and another glycosidase enzymes or mixtures act on polysaccharides, such as cellulose and hemicellulose, to convert all or a portion thereof to soluble sugars such as glucose, cellobiose, cellodextrins, xylose, arabinose, galactose, mannose or mixtures thereof. The soluble sugars may be predominantly cellobiose and glucose. The activity of cellulase enzyme mixtures produced by the fermentation process of the present invention in the hydrolysis of pre-treated lignocellulose is shown in Table 1.

The enzymatic hydrolysis is carried out at a pH and temperature that is at or near the optimum for the cellulase enzymes mixture produced by the fermentation process of the present invention. For example, the enzymatic hydrolysis may be carried out at about 30° C. to about 75° C., or any temperature therebetween, for example a temperature of 30, 35, 40, 45, 50, 55, 60, 65, 70, 75° C., or any temperature therebetween, and a pH of about 3.5 to about 8.0, or any pH therebetween, for example a pH of 3.5, 4.0, 4.5, 5.0, 5.5, 6.0, 6.5, 7.0, 7.5, 8.0 or any pH therebetween.

The initial concentration of cellulose, prior to the start of enzymatic hydrolysis of the pretreated lignocellulose, is preferably about 0.01% (w/w) to about 20% (w/w), or any amount therebetween, for example 0.01, 0.05, 0.1, 0.5, 1, 2, 4, 6, 8, 10, 12, 14, 15, 18, 20% or any amount therebetween. The combined dosage of all cellulase enzymes may be about 0.001 to about 100 mg protein per gram cellulose, or any amount therebetween, for example 0.001, 0.01, 0.1, 1, 5, 10, 15, 20, 25, 30, 40, 50, 60, 70, 80, 90, 100 mg protein per gram cellulose or any amount therebetween. The enzymatic hydrolysis of the pretreated lignocellulose may be carried out for a time period of about 0.5 hours to about 200 hours, or any time therebetween, for example, the hydrolysis may be carried out for a period of 2 hours to 100 hours, or any time therebetween, or it may be carried out for 0.5, 1, 2, 5, 7, 10, 12, 14, 15, 20, 25, 30, 35, 40, 45, 50, 55, 60, 65, 70, 75, 80, 85, 90, 95, 100, 120, 140, 160, 180, 200 hours or any time therebetween. The enzymatic hydrolysis of the pretreated lignocellulose may be batch hydrolysis, continuous hydrolysis, or a combination thereof. The hydrolysis may be agitated, unmixed, or a combination thereof. The enzymatic hydrolysis is typically carried out in a hydrolysis reactor. The cellulase enzyme may be added to the pretreated lignocellulosic substrate prior to, during, or after the addition of the substrate to the hydrolysis reactor. It should be appreciated that the reaction conditions are not meant to limit the invention in any manner and may be adjusted as desired by those of skill in the art.

### Treatment of Textiles Products

The cellulase enzyme mixture produced by the fermentation process of the present invention may be used to treat “textiles products” or “cellulose-containing goods”. Such treatments include “depilling” or “bio-stoning”.

The terms “textiles products” and “cellulose-containing goods” refers to fabrics, either as piece goods or goods sewn into garments or yarn, comprising cotton or non-cotton containing fibres.

As used herein, the term “depilling” refers to the use of a cellulase enzyme mixture produced by the fermentation process of the present invention in a controlled hydrolysis of cellulosic fibres in order to modify the surface of the cotton goods in a manner that clears the surface structure by reducing fuzzing. Such treatment can prevent pilling, improve fabric handling like softness and smoothness, which can result in clarification of colour and/or improve moisture adsorbability and dyeability. Depilling treatments typically provide agitation and shear to the fabric, including loose fibrils. In addition to cellulase enzyme and fabric, other components may be added during depilling, including water, buffer, detergents or surfactants.

In a typical depilling process, the treatment time may be between about 10 to about 120 minutes; treatment temperature may be about 20° C. to about 70° C.; the ratio of liquor to fabric may be between about 2.5:1 and about 10:1 by weight; and the pH may be about 4.0 to about 9.5. The amount of cellulase enzyme mixture used depends on the concentration of active protein in the mixture, the amount of cotton goods being treated, the desired degree of depilling, the time of treatment and other parameters well-known to those of ordinary skill in the art.

A cellulase enzyme mixture produced by the fermentation process of the present invention may also be used in a “bio-stoning” process. “Bio-stoning”, as used herein, refers to the use of enzymes in place of, or in addition to, pumice stones for the treatment of fabric or garments, especially denim. Bio-stoning typically has three steps: desizing, abrasion and after-treatment. Desizing involves removal of starch or other sizing agents usually applied to the warp yarns to prevent damage during the weaving process. Alpha-amylases can be used for such purpose. Abrasion may be performed with a cellulase enzyme mixture produced by the fermentation process of the present invention, either alone or together with pumice stones.

Bio-stoning treatment is usually carried out in washing machines, like drum washers. The pH of the abrasion reaction may range from 5-8 and the temperature may range from about 30° C. to 80° C. The liquor ratio (the ratio of the volume of liquid per weight of fabric) may range from about 3:1 to 20:1 and the treatment time can range between 15 minutes to 90 minutes. As is known by one of skill in the art, suitable enzyme dosages for imparting a stone-washed appearance to the fabric depend on the desired result, on the treatment method, and on the activity of the enzyme product.

In summary, the present invention provides highly productive fermentation processes that produce cellulase enzymes mixtures from Myceliophthora and taxonomically equivalent genera.

The above description is not intended to limit the claimed invention in any manner. Furthermore, the discussed combination of features might not be absolutely necessary for the inventive solution.

### EXAMPLES

### Example 1

### Myceliophthora thermophila Strains

M. thermophila strain CF-404 is described in U.S. Pat. No. 8,236,551 and is a derivative of strain C1, initially classified as Chrysosporium lucknowense (U.S. Pat. Nos. 6,015,707, 5,811,381 and 6,573,086; US Pat. Pub. Nos. 2007/0238155, US 2008/0194005, US 2009/0099079; International Pat. Pub. Nos., WO 2008/073914 and WO 98/15633; Visser et al., 2011, Indust. Biotechnol. 7: 214-223). Strain C1 is deposited at the All-Russian Collection of Microorganisms of Russian Academy of Sciences (VKM), Bakhurhina St. 8, Moscow, Russia, 113184 as Chrysosporium lucknowense Garg 27K, (Accession No. VKM F-3500-D).

### Example 2

### Myceliophthora Cellulase Production Using Glucose as a Carbon Source

a. Inoculum Preparation

Inoculum preparations for the fermentations were carried out as follows: a frozen, one milliliter (1 mL) aliquot of CF-404 culture stored in 20% glycerol and 0.3% NaCl was thawed and added to a 1 L baffled Erlenmeyer flask containing 300 mL of sterile media (Table 2). Inoculum cultures were incubated at 35° C. and 150-200 rpm for 72 h on a rotary shaker.

TABLE 2
Medium for Inoculum Preparation.

| Ingredient | | |
|---|---|---|
| K₂HPO₄ anhydrous (500 g/l stock solution), ml | 1 | mL |
| FeSO₄*7H₂O) (7 g/l stock solution), ml | 1 | mL |
| MgSO₄*7H₂O | 0.3 | g/L |
| Corn Steep Solids (dry CSL) | 12.5 | g/L |
| Glucose monohydrate | 20 | g/L |
| CaCO₃ | 3.68 | g/L |
| Industrol DF204 * | 3 | mL/L |

All components were dissolved in a beaker and adjusted to pH 7.0 using 10M NaOH. Each inoculum flask contained 300 mL of this solution. Media was autoclaved at 121° C. for 30 minutes.

b. Protein Production in a 5 L Fermentor

Inoculum cultures of strain CF-404 were used to inoculate 3.5 L of Cellulose-Free Initial Batch-phase Medium (Table 3) contained in three 5 L fermentors. For both the batch and subsequent fed-batch phases, the culture pH set point was maintained by the addition of a 10% NH₄OH solution. Aeration was accomplished by the addition of 4 slpm air and agitation by Rushton impellers at 750 rpm. The temperature was maintained at 38° C. The batch phase of the fermentation was allowed to proceed until all initial glucose was exhausted; the pH set point was 4.25. At this point the fed-batch phase began during which a solution of glucose was added at an average feed rate of 0.3-0.6 g carbon/L/h; the pH set point was 5.0. The combined duration of the batch and fed-batch phase was approximately 167 hours.

TABLE 3
Cellulose-Free Initial Batch-phase Medium

| Ingredient | Quantity (g/L) |
|---|---|
| (NH₄)₂SO₄ | 9.33 |
| MgSO₄*7H₂O | 0.49 |
| CaCl₂ | 0.30 |
| KH₂PO₄ | 1.52 |
| KCl | 0.52 |
| Glucose monohydrate | 13.0 |
| Corn Steep Solids | 30.0 |
| Biotin | 0.000006 |
| EDTA | 0.05 |
| ZnSO₄ * 7H₂O | 0.022 |
| H₃BO₄ | 0.011 |
| MnSO₄ * H₂O | 0.0043 |
| FeSO₄ * 7H₂O | 0.005 |
| CoCl₄* H₂O | 0.0017 |
| CuSO₄ * 5H₂O | 0.0016 |
| Na₂MoO₄ * 2H₂O | 0.0015 |
| Industrol DF204 | 3.0 |

All components were mixed in tap water and sterilized at 121° C. for 30 minutes. The pH was adjusted to 4.25 using NH₄OH.

c. Protein Production in a 14 L Fermentor

Inoculum cultures of strain CF-404 were prepared as described in Example 2a and used to inoculate 10 L of Cellulose-Free Initial Batch-phase Medium (Table 3) contained in three 14 L fermentors. For both the batch and subsequent fed-batch phases, the culture pH set point was maintained by the addition of a 10% NH₄OH solution. Aeration was accomplished by the addition of 8 slpm air and agitation by Rushton impellers at 500 rpm. The temperature was maintained at 34° C. The batch phase of the fermentation was allowed to proceed until all initial glucose was exhausted; the pH set point was 4.25. At this point the fed-batch phase began during which a solution of glucose was added at an average feed rate of 0.3-0.6 g carbon/L/h; the pH set point was 5.0. The combined duration of the batch and fed-batch phase was approximately 167 hours.

d. Recovery and Characterization of Culture Filtrates

The fungal mycelia and suspended solids were removed by filtration using a Buchner funnel, Fisherbrand®G6 glass fiber filter circles, and a vacuum apparatus. The culture filtrates were collected and assayed for protein concentration and cellulase activity.

The concentration of protein in the culture filtrate was determined using a variation of the Bradford method (Bradford, M. M. (1976) Analytical Biochemistry 72: 248-254). Using distilled water, several dilutions of the sample and a standard protein (of known quality and quantity) were prepared, ranging in concentration from approximately 0.1 to 1.0 g/L. For each dilution, a 0.2 mL aliquot was transferred to a test tube and combined with 2.0 mL of dye solution (0.057 g/L Coomassie Brilliant Blue G-250, 2.86% w/v methanol, 9.7% w/v phosphoric acid). After a 30 minute incubation at room temperature, changes in colour intensity were quantified spectrophotometrically using absorbance measurements at 595 nm. The protein concentration of the sample was calculated by plotting the absorbance signals against those of the control and accounting for the dilution factors.

The cellulase activity of the of the culture filtrates were determined using a filter paper activity (FPA) assay based on the standard cellulase activity measurement (Ghose, T. K. (1987) Pure & Appl. Chem. 59(2):257-268). This assay measures the combined activity of a whole cellulase on a crystalline substrate (Whatman® No. 1 filter paper). The activity is defined as the amount of enzyme required to produce 2.0 mg of glucose (measured as reducing sugar, or RS) from 0.05 g substrate in 60 minutes, under the described assay conditions (1.5 mL of 50 mM citrate buffer, pH 4.8 and 50° C.). FPA is reported in units of μmole of glucose/minute/mL of enzyme. The best linearization of the data to calculate enzyme activity is to use data that spans the range of 0.75 mg to 2.5 mg glucose produced and to plot the data as log RS produced versus log ml enzyme. Using linear regression, a slope and intercept are calculated for this data and activity is calculated using the following equation:

$$FPU / mL = \frac{2.0 {mg}_{glucose}}{180 μg \text{/} μmole} * \frac{1000 μg}{mg} * \frac{1}{60 min} * \frac{1}{0.5 {mL}_{enzyme }} * dilution factor$$

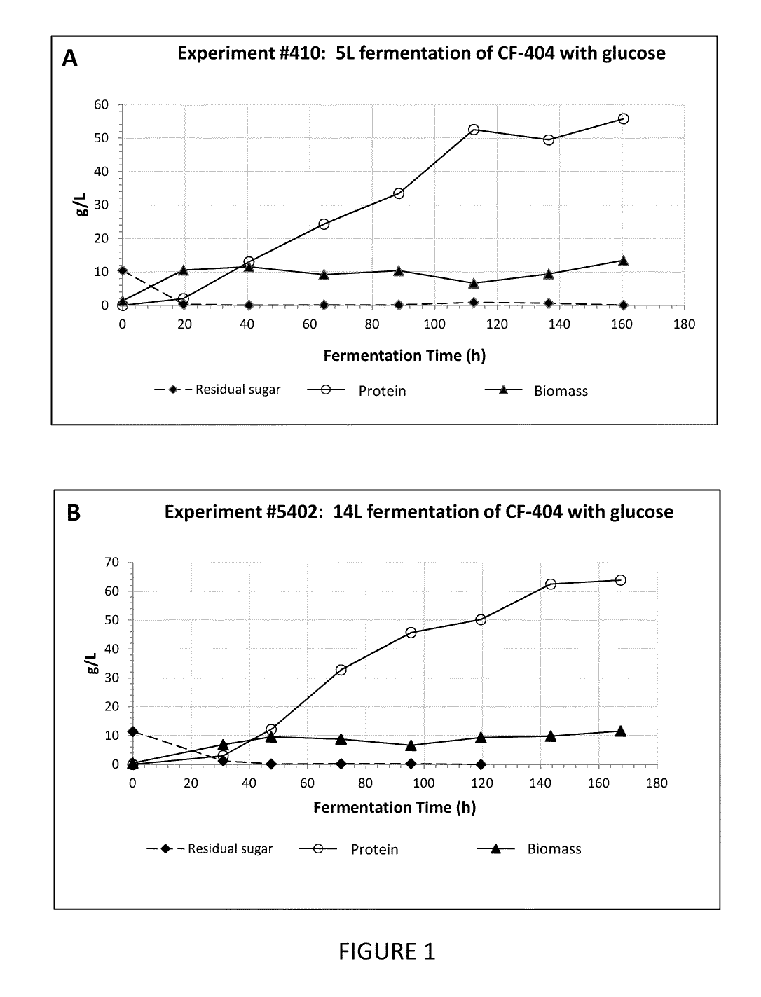

A profile of the accumulation of protein in the culture filtrates v. hours of fermentation is shown in FIG. 1. The total protein concentration and cellulase activity of M. thermophila cellulase mixtures in culture filtrates produced in fed-batch fermentations using only glucose as a carbon source are shown in Table 4. At both the 5 L and 14 L scale, the total protein concentrations and cellulase activity of these cultures filtrates are similar to those of filtrates from the cultures using glucose+inducing disaccharides as a carbon source (Tables 1 and 5); FIG. 1).

TABLE 4
Protein concentration and activity of M. thermophila cellulase mixtures
produced using glucose as carbon source

| | | Protein Concentration | Filter Paper Activity |
|---|---|---|---|
| Scale | Experiment # | (g/L) | (FPU/mL) |
| 5 L | 404 | 48.5 | 16.5 |
| | 406 | 38.6 | 13.3 |
| | 410 | 55.8 | 16.4 |
| 14 L | 5371 | 51.8 | 16.3 |
| | 5395 | 61.8 | 20.9 |
| | 5402 | 63.9 | 16.2 |

### Example 3

### Myceliophthora Cellulase Production Using a Mixture of Glucose and Inducing Disaccharides as a Carbon Source

Triplicate 5 L fed-batch fermentation was conducted as in Example 2b, except that the feed solution used for the fed-batch phase was a solution of glucose and various disaccharides. The ratio of the carbon s in the feed solution is glucose:cellobiose:gentiobiose:sophorose 1.00:0.13:0.05:0.02. After 167 hours, the culture filtrates were collected and characterized as described in Example 2d.

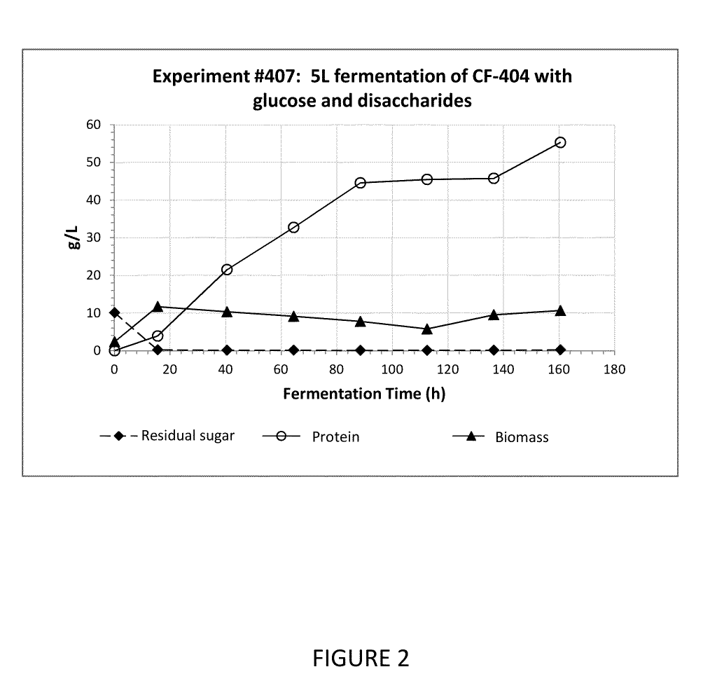

A profile of the accumulation of protein in the culture filtrate v. hours of fermentation is shown in FIG. 2. The total protein concentration and cellulase activity of M. thermophila cellulase mixtures in culture filtrates produced in fed-batch fermentations using a mixture of glucose and inducing disaccharides as a carbon source are shown in Table 5.

TABLE 5
Protein concentration and activity of M. thermophila cellulase mixtures
produced using a mixture of glucose and inducing disaccharides
as carbon source

| | Protein Concentration | Filter Paper Activity |
|---|---|---|
| Experiment # | (g/L) | (FPU/mL) |
| 405 | 41.3 | 17.0 |
| 407 | 55.3 | 18.3 |
| 411 | 47.2 | 17.3 |

### Example 4

### Myceliophthora Cellulase Production Using Glycerol as a Carbon Source

Triplicate 14 L fed-batch fermentations were conducted as in Example 2c, except that a solution of glycerol was used as the feed solution. After 167 hours, the culture filtrates were collected and characterized as described in Example 2d.

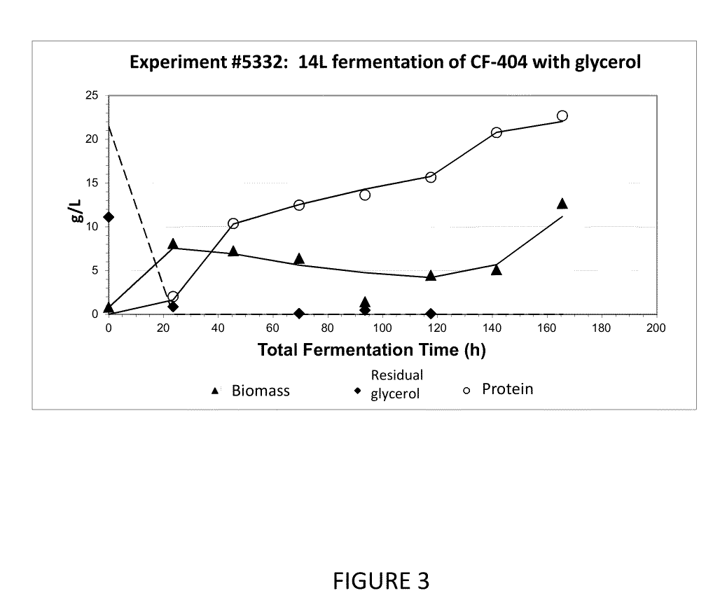

A profile of the accumulation of protein in the culture filtrate v. hours of fermentation is shown in FIG. 3. The total protein concentration and cellulase activity of M. thermophila cellulase mixtures in culture filtrates produced in fed-batch fermentations using only glycerol as a carbon source are shown in Table 6.

TABLE 6
Protein concentration and activity of M. thermophila cellulase mixtures
produced using glycerol as carbon source

| | Protein Concentration | Filter Paper Activity |
|---|---|---|
| Experiment # | (g/L) | (FPU/mL) |
| 5331 | 27.0 | 7.3 |
| 5332 | 31.5 | 9.2 |
| 5348 | 29.4 | 6.9 |

### Example 5

### Myceliophthora Cellulase Production Using Pure Xylose or a Mixture of Glucose and Xylose as a Carbon Source

Triplicate 5 L fed-batch fermentation was conducted as in Example 2b, except that the feed solution used for the fed-batch phase was either a solution of xylose or a solution of 90:10 (w:w) glucose:xylose. After 167 hours, the culture filtrates were collected and characterized as described in Example 2d.

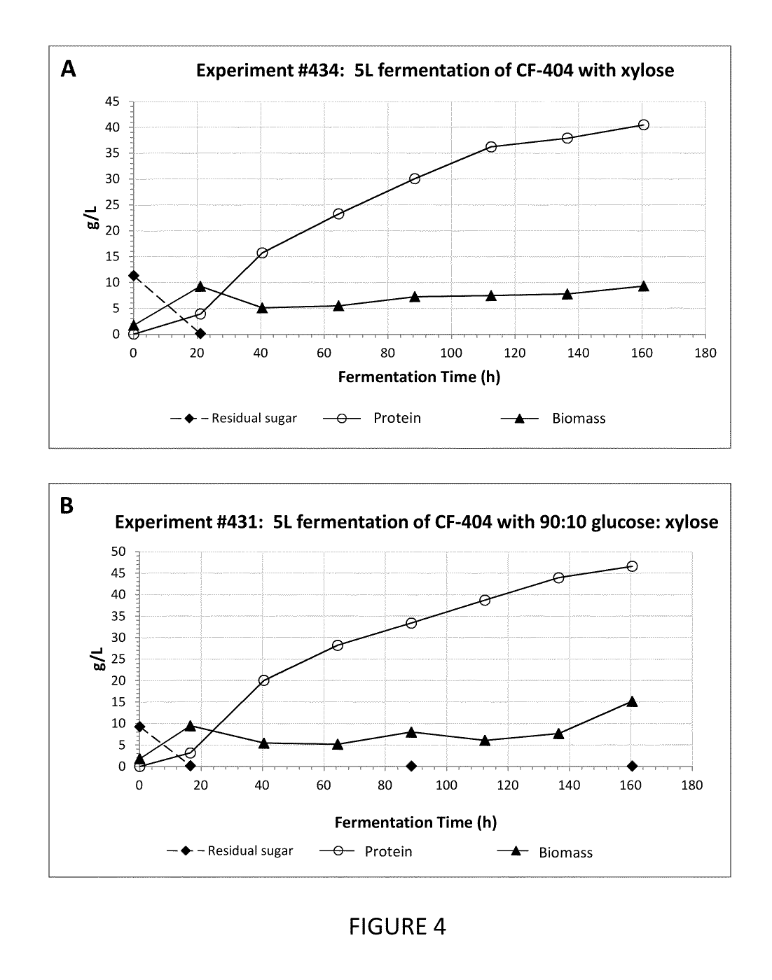

A profile of the accumulation of protein in the culture filtrate v. hours of fermentation is shown in FIGS. 4A and 4B. The total protein concentration and cellulase activity of M. thermophila cellulase mixtures in culture filtrates produced in fed-batch fermentations using pure xylose or a mixture of glucose and xylose as a carbon source are shown in Table 7.

TABLE 7
Protein concentration and activity of M. thermophila cellulase mixtures
produced using xylose or a mixture of glucose and xylose as carbon source

| | | | Filter |
|---|---|---|---|
| | | Protein | Paper |
| | | Concentration | Activity |
| Experiment # | Carbon Source in Feed | (g/L) | (FPU/mL) |
| 432 | xylose | 47.5 | 20.4 |
| 433 | | 39.7 | 26.2 |
| 434 | | 40.5 | 22.7 |
| 428 | 90:10 (w:w) glucose:xylose | 42.5 | 24.2 |
| 429 | | 38.8 | 21.3 |
| 431 | | 46.6 | 23.3 |

### Example 6

### Myceliophthora Cellulase Production Using Sucrose as a Carbon Source

Triplicate 14 L fed-batch fermentations were conducted as in Example 2c, except that a solution of sucrose was used as the feed solution. After 167 hours, the culture filtrates were collected and characterized as described in Example 2d.

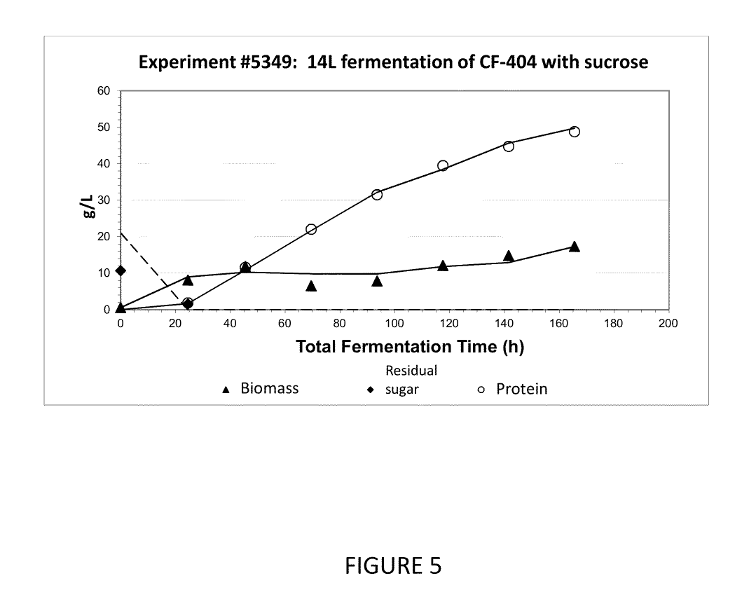

A profile of the accumulation of protein in the culture filtrate v. hours of fermentation is shown in FIG. 5. The total protein concentration and cellulase activity of M. thermophila cellulase mixtures in culture filtrates produced in fed-batch fermentations using only sucrose as a carbon source are shown in Table 8. The total protein concentrations and cellulase activities of these cultures filtrates are similar to those of filtrates from the cultures using glucose+inducing disaccharides as a carbon source (Tables 1 and 5; FIG. 2).

TABLE 8
Protein concentration and activity of M. thermophila cellulase mixtures
produced using sucrose as carbon source

| | Protein Concentration | Filter Paper Activity |
|---|---|---|
| Experiment # | (g/L) | (FPU/mL) |
| 5349 | 67.8 | 21.1 |
| 5372 | 57.8 | 14.1 |
| 5403 | 62.2 | 13.9 |

### Example 7

### Myceliophthora Cellulase Production Using a Molasses as a Carbon Source

Triplicate 14 L fed-batch fermentations were conducted as in Example 2c, except that a solution of molasses was used as the feed solution. After 167 hours, the culture filtrates were collected and characterized as described in Example 2d.

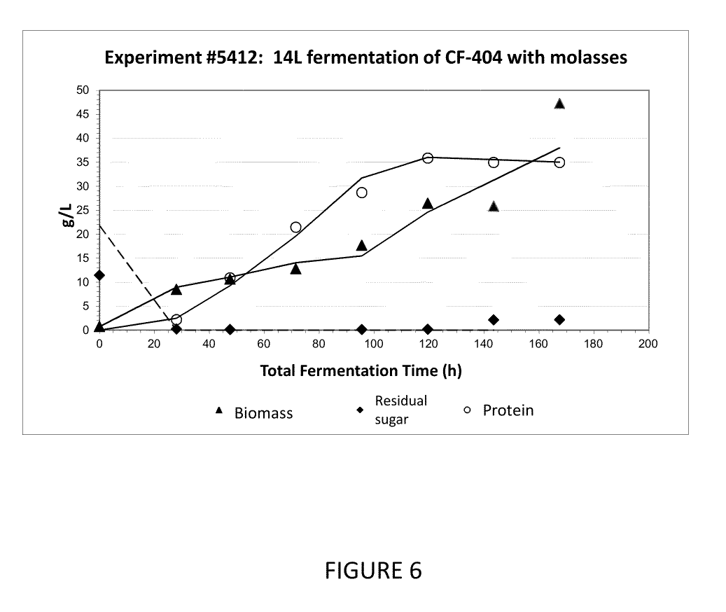

A profile of the accumulation of protein in the culture filtrate v. hours of fermentation is shown in FIG. 6. The total protein concentration and cellulase activity of M. thermophila cellulase mixtures in culture filtrates produced in fed-batch fermentations using only molasses as a carbon source are shown in Table 8. The total protein concentrations and cellulase activities of these cultures filtrates are similar to those of filtrates from the cultures using glucose+inducing disaccharides as a carbon source (Tables 1 and 7; FIG. 1).

TABLE 9
Protein concentration and activity of M. thermophila cellulase mixtures
produced using molasses as carbon source

| | Protein Concentration | Filter Paper Activity |
|---|---|---|
| Experiment # | (g/L) | (FPU/mL) |
| 5411 | 47.9 | 14.9 |
| 5412 | 48.5 | 15.9 |
| 5413 | 40.4 | 13.8 |

### Example 8

### Myceliophthora Cellulase Production Using Pure Fructose or a Mixture of Glucose and Fructose as a Carbon Source

Triplicate 14 L fed-batch fermentation was conducted as in Example 2c, except that the feed solution used for the fed-batch phase was either a solution of fructose or a solution of 50:50 (w:w) glucose:fructose. After 167 hours, the culture filtrates were collected and characterized as described in Example 2d.

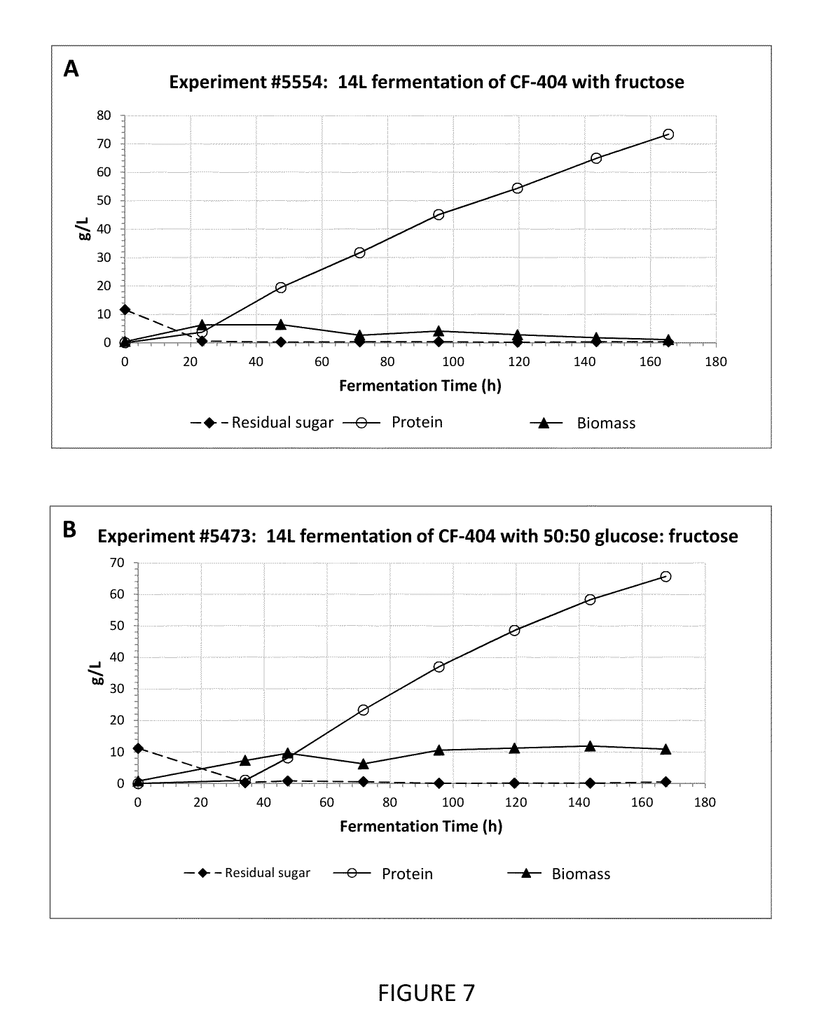

A profile of the accumulation of protein in the culture filtrate v. hours of fermentation is shown in FIGS. 7A and 7B. The total protein concentration of M. thermophila cellulase mixtures in culture filtrates produced in fed-batch fermentations using pure fructose or a 50:50 (w/w) mixture of glucose: fructose as a carbon source are shown in Table 10.

TABLE 10
Protein concentration of M. thermophila cellulase mixtures produced using
fructose or a mixture of glucose and fructose as carbon source

| | | Protein Concentration |
|---|---|---|
| Experiment # | Carbon Source in Feed | (g/L) |
| 5508 | 100 wt % fructose | 68.48 |
| 5554 | | 65.64 |
| 5555 | | 67.70 |
| 5472 | 50:50 (w:w) | 67.59 |
| 5473 | glucose:fructose | 73.36 |
| 5507 | | 74.67 |

1. A fermentation process for producing a cellulase enzyme mixture comprising,
(a) providing a fungal cell of the genus Myceliophthora or a taxonomically equivalent genus;
(b) culturing the fungal cell in a submerged liquid batch culture comprising a carbon source, wherein the carbon source is not cellulose;
(c) culturing the fungal cell from the batch culture from step (b) in a submerged liquid fed-batch, continuous, or combined fed-batch and continuous culture; and
(d) providing the culture of step (c) with a feed solution comprising a carbon source, wherein about 100 wt % of the carbon source is a non-inducing carbon source, and wherein the feed solution is provided at a rate that maintains the concentration of the non-inducing carbon source in the culture below that which would otherwise repress production of the cellulase enzyme mixture,
the fermentation process producing a culture filtrate containing the cellulase enzyme mixture at a concentration of at least 10 g protein/L filtrate.

2. The fermentation process of claim 1, wherein the non-inducing carbon source in the feed solution of step (d) is selected from the group consisting of glucose, dextrose, sucrose, molasses, fructose, glycerol, xylose, and any combination thereof.

3. The fermentation process of claim 1, wherein the feed solution is provided at a rate that maintains the concentration of the non-inducing carbon source in the culture below 2 g/L.

4. The fermentation process of claim 1, wherein the step of culturing in step (c) comprises culturing the fungal cell from the batch culture in a submerged liquid fed-batch culture, and wherein the process produces a culture filtrate containing the cellulase enzyme mixture at a concentration of at least 25 g protein/L filtrate.

5. The fermentation process of claim 4, wherein the feed solution of step (d) is provided a rate of from about 0.2 to about 4 g carbon/L/h.

6. The fermentation process of claim 1, wherein the step of culturing in step (c) comprises culturing the fungal cell from the batch culture in a submerged liquid continuous culture, and wherein the process produces a culture filtrate containing the cellulase enzyme mixture at a concentration of at least 10 g protein/L filtrate at steady state.

7. The fermentation process of claim 6, wherein the dilution rate of the continuous culture is from about 0.001 to about 0.1 h⁻¹.

8. The fermentation process of claim 1, wherein the taxonomically equivalent genus is Sporotrichum, Thielavia, Chrysosporium, Corynascus or Ctenomyces.

9. The fermentation process of claim 1, wherein the fungal cell is a strain of Myceliophthora thermophile, Sporotrichum thermophile, Sporotrichum thermophilum, Sporotrichum cellulophilum, Thielavia heterothallica, Chrysosporium thermophile, Chrysosporium lucknowense, or Corynascus heterothallicus.

10. The fermentation process of claim 1, wherein the fungal cell comprises one or more mutations that result in production of a cellulase enzyme mixture in the absence of a cellulase-inducing carbon source.

11. The fermentation process of claim 1, wherein the fungal cell comprises one or more mutations that result in production of a cellulase enzyme mixture in the presence of a cellulase non-inducing carbon source.

12. The fermentation process of claim 1, wherein the fungal cell is genetically modified to enhance or reduce the expression or activity of one or more protein of interest selected from the group consisting of cellulase, hemicellulase, beta-glucosidase, esterase, cellulase-enhancing polypeptide, cellobiose dehydrogenase, laccase, lignin peroxidase, manganese peroxidase, beta-glucanase, protease, amylase, and glucoamylase.

13. The fermentation process of claim 12, wherein the protein of interest is homologous or heterologous with respect to the fungal cell.

14. A cellulase mixture produced by the fermentation process of claim 1.

15. A process for hydrolyzing cellulose comprising treating a cellulosic substrate with the cellulase enzyme mixture of claim 14.

16. The process of claim 15, wherein the cellulosic substrate is pretreated lignocellulose.

17. The process of claim 15, wherein the cellulosic substrate is a textiles product.

18. A fermentation process for producing a cellulase enzyme mixture comprising,
(a) providing a fungal cell of the genus Myceliophthora or a taxonomically equivalent genus;
(b) culturing the fungal cell in a submerged liquid batch culture comprising a carbon source, wherein the carbon source is not a cellulase-inducing carbon source;
(c) culturing the fungal cell from the batch culture from step (b) in a submerged liquid fed-batch, continuous, or combined fed-batch and continuous culture; and
(d) providing the culture of step (c) with a feed solution comprising a carbon source, wherein about 100 wt % of the carbon source is a non-inducing carbon source, and wherein the feed solution is provided at a rate that maintains the concentration of the non-inducing carbon source in the culture below that which would otherwise repress production of the cellulase enzyme mixture,
the fermentation process producing a culture filtrate containing the cellulase enzyme mixture at a concentration of at least 25 g protein/L filtrate.
